(19) United States
(12) Patent Application Publication
(10) Pub. No.: US 2007/0110676 A1
(43) Pub. Date: May 17, 2007

(54) Compositions useful for prevention and treatment of common cold and influenza-like symptoms

(75) Inventors: Jeffrey Warren Clymer, Mason, OH (US); Begonia Y. Ho, Cincinnati, OH (US); Mary Lynn Jump, Fairfield, OH (US); Leonard Edwin Small, Cincinnati, OH (US); Amy Ann Walanski, Fairfield, OH (US); Paul John Rennie, Godalming (GB); Claudine Killar Zukowski, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/282,010

(22) Filed: Nov. 17, 2005

(51) Int. Cl. A61K 9/12; A61K 36/906; A61K 36/28; A61K 33/32; A61K 31/192; A61K 31/4745; A61K 31/137

(52) U.S. Cl. 424/45; 424/737; 514/699; 514/717; 424/641; 514/494; 424/756; 514/569; 514/570; 514/649; 514/291; 514/424; 514/250

(57) ABSTRACT

The present invention is directed to compositions that are useful in the prevention and treatment of common cold and influenza-like symptoms due to respiratory tract viral infections. These compositions comprise a guaiacol component, and a mucoadhesive polymer wherein the pH of the composition is about 5.5 or less.

### FIELD OF THE INVENTION

The present invention is directed to compositions useful for the prevention and treatment of common cold and influenza-like symptoms due to respiratory tract viral infections, wherein these compositions are effective in preventing the onset of the symptoms of common cold and influenza or significantly mitigating them if an individual is already afflicted with such common cold and influenza-like symptoms

### BACKGROUND OF THE INVENTION

It is known that many different viruses and viral strains result in symptoms associated with respiratory viral infections. The common cold is a complex syndrome caused by over 200 antigenically different viruses found in five virus families. These families include rhinovirus, myxovirus, paramyxovirus, respiratory syncytial virus, adenovirus and coronavirus. The most important group, with respect to the common cold, is rhinovirus, Gwaltney J. M., Common cold, pp 489-493, Mandell G. L., Douglas, R. G. Jr., Bennett, J. E., Principles and Practice of Infectious Diseases, 3rd ed., Churchill Livingstone, New York, 1990. Pinpointing the specific cause of the illness is difficult and not practical since there are also a number of predisposing factors whose contribution to the manifestation of symptoms is not fully understood. Such include, but are not limited to, physical fatigue, psychological stress, and overall physical health.

Regardless of the virus and associated factors leading to the onset of common cold and influenza symptoms, a number of remedies to alleviate the symptoms of the common cold have been suggested. The cough/common cold products that are currently marketed typically contain one or more of the following actives: nasal decongestants such as pseudoephedrine or oxymetazoline, antihistamines such as doxylamine; antitussives such as dextromethorphan; expectorants such as guaifenesin; and anti-pyretics such as acetaminophen. In an attempt to improve existing common cold remedies, experts in the field have suggested several alternative pharmacotherapies and have conducted common cold trials to test their efficacy. Examples of these therapies include: the use of interferon, Douglas et al., Prophylactic Efficacy of Intranasal Alpha-Interferon Against Rhinovirus Infection in the Family Setting, The New England Journal of Medicine, 314, pp. 65-70, 1986; bradykinin antagonist, Higgins et al., A Study of the Efficacy of the Bradykinin Antagonist, NPC567; in Rhinovirus Infections in Human Volunteers, Antiviral Research vol. 14, pp. 339-344, 1990; glucocorticoid, Parr et al., A Randomized Controlled Trial of Glucocorticoid Prophylaxis Against Experimental Rhinovirus Infection, Journal of Infectious Diseases, vol. 162, pp. 1173-1177, 1990; nedocromil, Barrow et al., The Effect of Intranasal Nedocromil Sodium on Viral Upper Respiratory Tract Infections in Human Volunteers, Clinical and Experimental Allergy, vol. 20, pp. 45-51, 1990; a combination of interferon-α₂, ipratropium and naproxen, Gwaltney, Combined Antiviral and Antimediator Treatment of Rhinovirus Colds, The Journal of Infectious Diseases vol. 166, pp. 776-782, 1992; zinc salts, Potter et al., DIAS Rounds, Zinc Lozenges for Treatment of Common Colds, The Annals of Pharmacotherapy, vol. 27, pp. 589-592, 1993.

A number of patents have also been issued disclosing compositions for prevention and treatment of the common cold and their methods of use. A sample of such patents includes: U.S. Pat. Nos. 5,240,694; 5,422,097; and 5,492,689; all to Gwaltney, disclosing treatment using; combinations of anti-viral and anti-inflammatory compounds; U.S. Pat. Nos. Re 33,465 and 5,409,905; both to Eby disclosing treatment using zinc salts; U.S. Pat. No. 5,626,831; to Van Moerkerken disclosing treatments using orally administered aminocarboxylic acid compounds; U.S. Pat. Nos. 4,619,934 and 4,552,899, both to Sunshine, disclosing treatment of cough and common colds using compositions comprising non-steroidal anti-inflammatory drugs such as NSAIDS with antihistaminically effective materials such as chlorpheniramine. Treatment for influenza includes vaccination and use of specific antiviral drugs.

Amantidine and rimantidine have been used for treating influenza infections. They are reported to target the M2 protein of influenza virus and interfere with release of viral genetic material into the infected cell, thus preventing viral replication. A number of side effects have been reported including neurological and gastro-intestinal complaints, Beishe et al., Genetic basis of Resistance to Rimantidine Merging During Treatment of Influenza Virus Infection, Journal of Virology, 1988, 62, 1508-12; Hay, A. J., The Action of Adamantanamines Against Influenza A Viruses: Inhibition of the M2 Ion Channel, Protein. Semin Virol., 1992, 3, 21-30.

Another approach to influenza treatment has been to inhibit the neuraminidase enzyme molecule on the virus, important to the virus' replication and infectivity. One such treatment drug is Zanamivir. See Robinson et al., “Zanamivir in the prevention of influenza among healthy adults: A randomized controlled trial,” JAMA, 1999, 282, 31-5. Side effects reported were sinusitis, diarrhea, nausea, and adverse lung effects. A second neuraminidase inhibiting drug is Oseltamivir, licensed under the trade name Tamiflu. See Treanor et al., “Efficacy and safety of the oral neuraminidase inhibitor Oseltamivir in treating acute influenza: A randomized controlled study,” JAMA, 2000, 283, 1016-24. There is a concern with Amantidine, Rimantidine and Neuraminidase inhibitors that viral resistance may develop, rendering them ineffective, A. Elliot and J. Ellis, 2000, Pharmaceutical Journal, 265, 446-451.

U.S. Pat. No. 4,689,223, issued Aug. 25, 1987, assigned to T&R Chemicals, discloses nasal spray compositions for treating the symptoms of or preventing the common cold, wherein the compositions comprise sulphites or bisulphites having low pH, but no specific pH is disclosed.

EP046409, published Feb. 24, 1982, to Walliczek, discloses processes for the preparation of solutions of cuprous complexes for therapeutic treatment of human or animal body. One disclosed process includes the preparation of cuprous complex with ascorbic acid or non toxic ascorbate having a pH from 4-6. The complex may be used to make solutions for topically treating fungal, inflammatory or viral complaints. Other disclosures of nasal compositions comprising viscous gels or other viscosity building polymeric materials include U.S. Pat. No. 4,891,226; WO 91/10434; U.S. Pat. No. 4,478,822; U.S. Pat. No. 5,599,534; U.S. Pat. No. 5,215,739; U.S. Published Application 2002/0032231; U.S. Pat. No. 6,365,624; U.S. Pat. No. 5,158,761; U.S. Pat. No. 5,897,858; and EP 1108422.

Homeopathic ingredients, as specified in the Homeopathic Pharmacopeia of the U.S. and other compendia, have been evaluated in the treatment of the common cold, but with questionable success. One of the homeopathic ingredients most exhaustively studied is Echinacea. Various other non-homeopathic herbal and plant-derived compounds have been studied as well.

A known characteristic of rhinoviruses is that they lose infectivity under acidic conditions. Even pH values of 5.0 are known to reduce Rhinovirus infectivity, Hughes, J. H., Acid Lability of Rhinovirus Type 14: Effect of pH, Time and Temperature, Proc. Soc. Exp. Biol. Med., 1993, 144, 555-60. EP310317, published Apr. 5, 1989 to Bordt et al., assigned to Beecham, discloses a method for inactivating viruses and bacteria with pharmaceutical compositions including vaccines prepared by inactivating viruses or bacteria with ascorbic acid or its salts in the presence of oxygen and heavy metal ions. U.S. Pat. No. 4,767,788, Diana, issued Aug. 30, 1988, assigned to Sterling Drug Inc., discloses processes for destroying viruses with glutaric acid including rhinovirus in the nasal mucosa.

Therefore, although a low pH vehicle can potentially provide efficacy benefits to the delivery of medicaments, including homeopathic, allopathic or herbal drugs in the treatment of upper respiratory tract conditions, there are drawbacks to the aesthetic and pharmaceutical formulations of such vehicles. The low pH of the vehicle can produce irritation in the nasal cavity upon application. This effect may lead to reduced patient compliance and hence diminished efficacy of the formulation. Moreover, typical preservative systems for liquids are most effective at near neutral pH. Hence there are difficulties in maintaining the microbial integrity of low pH vehicles in addition to the perceived irritation of the nasal cavity.

Despite the abundance of treatments known in the art, there remains a need to provide a consistent and effective method for prevention or treatment of common cold or influenza-like symptoms. There also remains a need to provide compositions, particularly nasal compositions, that are highly effective in the prevention or treatment of common cold and influenza-like symptoms, wherein the compositions comprise a delivery vehicle that optimizes the efficacy of the medicaments for the improved prevention or treatment of these common cold and influenza-like symptoms.

### SUMMARY OF THE INVENTION

The present invention is directed to compositions useful for the prevention and treatment of common cold and influenza-like symptoms due to respiratory tract viral infections, wherein these compositions are effective in preventing the onset of the symptoms of common colds and influenza or significantly mitigating them if an individual is already afflicted with such symptoms.

In one embodiment herein, the invention is directed to compositions comprising a guaiacol component and a mucoadhesive polymer wherein the composition has a pH of about 5.5 or less.

In another embodiment herein, the invention is directed methods of treating or preventing a symptom associated with common cold or influenza in a mammal in need of such treatment or prevention, comprising administering to the mammal a composition comprising a guaiacol component and a mucoadhesive polymer, wherein the composition has a pH of about 5.5 or less. The subject mammal is a human, dog, cat, horse, goat, or any mammal that can be affected by the common cold, infected with influenza, or suffer from cold or influenza-like symptoms.

These and other aspects of the present invention are described in further detail herein.

### DETAILED DESCRIPTION OF THE INVENTION

Various documents including, for example, publications and patents, are recited throughout this disclosure. All such documents are hereby incorporated by reference.

All percentages and ratios are calculated by weight unless otherwise indicated. All percentages and ratios are calculated based on the total composition unless otherwise indicated.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments or individual features are disclosed. As will be apparent to the ordinarily skilled practitioner, all combinations of such embodiments and features are possible and can result in preferred executions of the present invention.

The compositions herein may comprise, consist essentially of, or consist of any of the elements as described herein.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

The compositions of the present invention are useful for the prevention or treatment of common cold or influenza-like symptoms.

As used herein “common cold and influenza-like symptoms” refer to one or more symptoms typically associated with respiratory tract viral infections. These symptoms include, but are not limited to, nasal congestion, chest congestion, sneezing, rhinorrhea, fatigue or malaise, coughing, fever, chills, body ache, sore throat, headache, and the like.

The term “respiratory viruses” as used herein refers to any of a variety of viruses that are causal agents of common cold and influenza-like symptoms. These viruses include, but are not limited to, Rhinovirus, Myxovirus (Influenza virus), Paramyxovirus (Parainfluenza virus), Respiratory Syncytial virus, Adenovirus and Coronavirus.

The term “safe and effective amount” of a component, composition, or like material as used herein is an amount that is effective for the prevention and treatment of common cold and influenza-like symptoms in a mammal (preferably a human), without undue adverse side effects (such as toxicity, irritation, or allergic response), commensurate with a reasonable benefit/risk ratio when used in the manner of this invention. The specific “safe and effective amount” will, obviously, vary with such factors as the particular condition being treated, the physical condition of the treated mammal, the size and weight of the treated mammal, the duration of treatment, the nature of concurrent therapy (if any), the specific dosage form to be used, other components present in a given dosed composition, and the dosage regimen desired for the component or composition.

Compositions and Components Used in the Present Invention

In one embodiment, the compositions herein comprise a guaiacol component, and a mucoadhesive polymer, wherein the composition has a pH of about 5.5 or less. Without intending to be limited by theory, it has been found that upon application to the nasal or other mucosal tissues, the compositions of the present invention create an environment hostile to respiratory viruses that permits the composition to function effectively. Such an environment deters viruses from infecting the nasal cavity. The compositions of the present invention are also suitable for treating mammals already infected with a respiratory virus in order to mitigate common cold and influenza-like symptoms, as well as being suitable for applications of reducing or eliminating the possibility of acquisition of such viruses when confronted with a high-risk public environment including schools, office buildings, public transit, and public events such as sporting or concert events. As previously stated, the compositions of the present invention are especially effective when administered in the preferred embodiment of a nasal composition, such as a nasal spray, comprising the combination of a guaiacol component and a mucoadhesive polymer wherein the composition has a pH of about 5.5 or less.

Again without being bound by theory, it is believed that the mucoadhesive polymer may provide for adherence of the composition to mucosal tissue and fluid, while the guaiacol component may remove the irritation often associated with nasally-applied compositions, and may alternatively or additionally improve the stability of the composition, to result in improved prevention and treatment of common cold and influenza-like symptoms.

The components of the present inventive compositions, as well as those components utilized in the methods herein (as described further below) are described as follows:

Guaiacol Component

The compositions herein further comprise a guaiacol component, defined herein as guaiacol or a 4-substituted derivative thereof. It is surprisingly discovered herein that the guaiacol component may be useful for reduction of irritation of mucosal tissue and/or improvement of the microbial efficacy of the composition itself, whether through actual enhanced efficacy and/or improved compliance when in use by the mammal under treatment.

As is commonly known in the art, guaiacol has the following chemical structure:

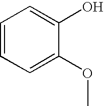

Guaiacol may be characterized as a yellowish, oily, aromatic substance derived from guaiacum or wood creosote, usable as an expectorant, a local anesthetic, or an antiseptic.

As defined herein, the guaiacol component may also be a 4-substituted derivative of guaiacol, which derivatives will be commonly understood in the art. The 4-substituted derivatives of guaiacol are well-known in the art, many of which are natural products or products derivable from such natural products. As a non-limiting example, the 4-substituted derivatives of guaiacol may have the following chemical structure: 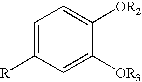
wherein R is selected from the group consisting of hydroxy, C₁-C₆ alkyl, C₁-C₆ alkenyl, C₁-C₆ alkoxy (e.g., methoxy), C₁-C₆ ethers (e.g., methyl propoxy), C₁-C₆ aldehydes, and C₁-C₆ ketones; wherein OR₂ is selected from the group consisting of hydroxy, C₁-C₆ esters (e.g., acetate) and C₁-C₆ alkoxy (e.g., methoxy); and wherein OR₃ is selected from the group consisting of C₁-C₆ esters (e.g., acetate) and C₁-C₆ alkoxy (e.g., methoxy).

Non-limiting examples of such 4-substituted derivatives of guaiacol include eugenol, iso-eugenol, dihydroeugenol, vanillyl butyl ether, vanillin (4-formyl-guaiacol), 5-propenylguaethol, 4-ethyl-2-methoxyphenol, 4-allyl-2-methoxyphenol acetate, and 4-methyl guaiacol. In one embodiment, the 4-substituted derivative of guaiacol is eugenol.

The guaiacol component may be a component mixture containing guaiacol or a 4-substituted derivative thereof. Such component mixtures are advantageously natural products, or products derived from natural products, that have relatively high content of the guaiacol or 4-substituted derivative thereof. As an example, cassia, clove, and cinnamon each contain guaiacol or 4-substituted derivatives thereof, or mixtures thereof. As such, essential oils, extracts or any product derived from cassia, clove, cinnamon, or any mixture thereof can be used as the guaiacol component herein. Essential oils of cassia, clove, or cinnamon may be particularly useful. Clove oil may be particularly useful. As an example, products derived from cassia, clove or cinnamon may contain eugenol at useful levels.

Without limitation by theory, the guaiacol component may improve the microbial integrity of low pH compositions. There is often difficulty in preserving liquids at low pH from microbial contamination, as most preservative systems are designed to work at near neutral pH. In some cases preservative systems may not function at low pH because of ionization of the active principal, or because the active principal is inherently unstable at low pH. The guaiacol component may not ionize at moderately low pH and are in general stable for reasonable periods of time at moderately low pH. Furthermore, a low pH composition containing a guaiacol component may have unexpectedly good microbial preservation properties, allowing such composition to be manufactured in a product with an adequate shelf life.

The guaiacol component may optionally be included at concentrations ranging from about 0.0001% to about 1% by weight, alternatively from about 0.001% to about 0.5% by weight, alternatively about 0.001% to about 0.07% by weight, alternatively from about 0.001% to about 0.02% by weight.

Mucoadhesive Polymer

The compositions of the present invention further comprise a mucoadhesive polymer. Without intending to be limited by theory, the mucoadhesive polymer may provide improved retention of the composition in areas of the respiratory tract such as the nasal cavity or other mucosal tissues, resulting in improved prevention or treatment of common cold and influenza-like symptoms with reduced nasal irritation.

The mucoadhesive polymer suitable for use herein includes any solid or liquid used alone or in combination to impart a change in the viscosity of the compositions upon contact of the compositions with stimulus such as pH, body temperature, change in ionic concentration, and the like. Therefore, mucoadhesive polymers are sometimes commonly referred to as viscosity building polymers. It is found that the incorporation of a mucoadhesive polymer that provides for a change in viscosity results in reducing and/or eliminating perceived irritation, especially perceived nasal irritation that can be caused by low pH conditions.

The mucoadhesive polymer suitable for use herein provides for the adherence of the compositions to mucosal tissues, particularly nasal mucosal tissues, such that the composition comes into contact with mucosal tissues and fluids to result in a change in viscosity of the composition in the respiratory tract or other mucosal areas. As a result, the compositions of the present invention are maintained on the mucosal surface for periods longer than typical respiratory tract compositions, thus maintaining a virus-hostile environment for the improved prevention and treatment of common cold and influenza-like symptoms. For example, wherein the compositions of the present invention are administered as a liquid composition, the composition may be applied using an atomizing sprayer, and upon spraying into a respiratory tract area such as the nasal cavity the composition may form a polymeric film, preferably a polymeric viscous film, that adheres to the nasal or other mucosal tissues. The polymeric film is may be a thin polymer film, such as a thin polymeric viscous film, that is also resistant to erosion upon sneezing, blowing of the nose, or upon mucociliary clearance. The mucoadhesive polymer is also capable of changing the viscosity of the compositions in situ upon application of the compositions to the mucosal tissues and fluids.

Known mucoadhesive polymers suitable for use herein include but are not limited to carboxypolymethylenes, carboxyvinyl polymers, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, natural polymers, polymeric thermoreversible polymers, ionic responsive polymers, copolymers of polymethyl vinyl ether and maleic anhydride, and mixtures thereof.

Nonlimiting examples of suitable homopolymers of acrylic acid crosslinked with an allyl ether of pentaerythritol or an allyl ether of sucrose are available from B. F. Goodrich Company under the tradename CARBOPOL Specific CARBOPOLS include CARBOPOLS 934, 940, 941, 956, 980, and mixtures thereof. CARBOPOLS 980 is preferred among the CARBOPOLS mucoadhesive polymers. Polymers of this type have slightly acidic carboxyl group substituents. Such polymers generally have a pH of about 3 in water and are generally used by neutralization during preparation of compositions to form viscous films and/or gels. When the respiratory tract compositions of the present invention comprise one or more CARBOPOLS mucoadhesive polymers, generally these polymers are used at concentrations ranging from about 0.01% to about 2.5% by weight of the composition.

Nonlimiting examples of suitable homopolymers of acrylic acid crosslinked with divinyl glycol are available from B. F. Goodrich Company as polycarbophils under the tradename NOVEON. Other nonlimiting examples of a mucoadhesive polymer suitable for use herein include natural polymers, polymeric cellulose derivatives, polyvinyl pyrrolidones (PVPs), dextran polymers, polyethylene oxide polymers including Polyox-600, thermoreversible polymers, ionic responsive polymers, copolymers of polymethyl vinyl ether and maleic anhydride, and mixtures thereof. For example, polymeric cellulose derivatives and thermoreversible polymers may be used.

Specific nonlimiting examples of natural polymers suitable for use as a mucoadllesive polymer herein include arabic gums, tragacanth gums, agar polymers, xanthan gums, copolymers of alginic acid and sodium alginate, chitosan polymers, pectins, carageenans, pollulan polymers, modified starches, and mixtures thereof.

Specific nonlimiting examples of polymeric cellulose derivatives suitable for use as a preferred mucoadhesive polymer herein include hydroxy alkyl cellulose polymers including hydroxypropyl methylcellulose (HPMC) and hydroxypropyl cellulose (HPC), methyl cellulose polymers, carboxymethyl cellulose (CMC) polymers, salts of carboxymethyl cellulose including sodium salt of carboxymethyl cellulose, and mixtures thereof.

Specifc nonlimiting examples of thermoreversible polymers suitable for use as a preferred mucoadhesive polymer herein include poloxamers including, for example, polyethylene-polypropylene glycols. Non-limiting examples include those commercially available as LUTROL F-127 (alpha-hydro-omega-hydroxypoly(oxyethylene)a poly(oxypropy-lene)b poly(oxyethylene)a block copolymer or polyethylene-polypropylene glycol) (polaxamer 407) and LUTROL F-68 (polaxamer 188), LUTROL F-108 (polaxamer 338) (commercially available from BASF), ethylhydroxy ethylcellulose (EHEC), and mixtures thereof. Polyethylene-polypropylene glycols are particularly useful herein.

Specific nonlimiting examples of ionic responsive polymers suitable for use as a mucoadhesive polymer herein include gelrite, gellan gum, KELCOGEL F and mixtures thereof.

Specific nonlimiting examples of copolymers of polymethyl vinyl ether and maleic anhydride suitable for use as a mucoadhesive polymer herein include such copolymers commercially available under the GANTREZ tradename including GANTREZ S and GANTREZ MS type copolymers.

Further examples of the mucoadhesive polymer suitable for use herein are even further described in the Journal Pharmacy Pharmacology 537 pages 3-22, (2001 Edition); the International Journal of Pharmaceutics (1988, 1996 and 1998 Editions); and the Journal Controlled Release 62, pages 101 107, (1999 Edition).

The mucoadhesive polymer can be included in the compositions of the present invention as an individual mucoadhesive polymer or as a combination of mucoadhesive polymers. The compositions may comprise from about 0.01% to about 30%, alternatively from about 0.1% to about 20%, alternatively from about 1% to about 15%, by weight of the composition, of total mucoadhesive polymer.

The incorporation of the mucoadhesive polymer into the compositions of the present invention may result in a composition that has a viscosity in the range of from about 1 centipoise (cps) to about 2000 cps, alternatively from about 1 cps to about 1000 cps, alternatively from about 5 cps to about 500 cps, and alternatively from about 5 cps to about 300 cps.

The viscosity of the compositions of the present invention is determined using the known methods described in ASTM D1824-87, ASTM D1084-88, and ASTM D2196-86.

pH

The compositions herein have a pH of about 5.5 or less, alternatively about 4.5 or less. The desired pH of the compositions of the present invention is achieved by using at least one acid. Without intending to be limited by theory, it is believed that an acid enables a composition to be produced that is hostile to viruses known to contribute to common cold and influenza-like symptoms.

In one embodiment, one or more organic acids can be used. Non-limiting examples of organic acids suitable for use herein include: ascorbic acid, monocarboxylic acids, dicarboxylic acids, tricarboxylic acids, and mixtures thereof. Specific non-limiting examples of suitable monocarboxylic, dicarboxylic, or tricarboxylic acids include salicylic, fumaric, benzoic, glutaric, lactic, citric, malonic, acetic, glycolic, malic, adipic, succinic, aspartic, phthalic, tartaric, glutamic, gluconic, and mixtures thereof.

In one embodiment, the acid has a dissociation constant (pKa) of from about 3.0 to about 5.5.

The acid suitable for use herein can be included in the compositions as an individual acid or as a combination of acids. In one embodiment, compositions comprise from about 0.01% to about 10%, alternatively from about 0.05% to about 5%, alternatively from about 0.1% to about 2.5%, of organic acid, by weight of the composition (wherein the percentages are for total organic acid less any additional acid present in the composition).

In one embodiment herein, the compositions optionally further comprise pyroglutamic acid. The composition can comprise pyroglutamic acid in combination with at least one other acid, for example an organic acid. Without intending to be limited by theory, it is believed that compositions comprising an organic acid in combination with pyroglutamic acid may have an increased buffering capacity. In one embodiment, this increased buffering capacity may enable the composition to achieve and maintain a surface pH of the tissue treated in the nasal cavities or turbinates of from about 3 to about 5.5.

As used herein, the pyroglutamic acid suitable for use in the compositions of the present invention includes those pyroglutamic acids collectively referred to as stereoisomers and tautomers of pyroglutamic acid. As also used herein, the pyroglutamic acid also includes its pharmaceutically acceptable salts.

For example, pyroglutamic acid, which is also referred to as pyrrolidone carboxylic acid, has two stereoisomers (D and L) and each are preferred for use herein. The D stereoisomer of pyroglutamic acid is also known by the following names: D-Proline, 5-oxo-(+)-2-Pyrrolidone-5-carboxylic acid, (+)-Pyroglutamic acid, (R)-2-Pyrrolidone-5 carboxylic acid, 5-Oxo-D-proline, D-2-Pyrrolidone-5-carboxylic acid, D-Pyroglutamic acid, D-I Pyrrolidinonecarboxylic acid, and D-Pyrrolidonecarboxylic acid.

The L stereoisomer of pyroglutamic acid is also known by the following names: L Proline, 5-oxo-(−)-2-Pyrrolidone-5-carboxylic acid, (−)-Pyroglutamic acid, (5S)-2-Oxopyrrolidine-5-carboxylic acid, (S)-(−)-2-Pyrrolidone-5-carboxylic acid, (S)-2-Pyrrolidone-5-carboxylic acid, (S)-5-Oxo-2-pyrrolidinecarboxylic acid, (S)-Pyroglutamic acid, 2-L-Pyrrolidone-5-carboxylic acid, 2-Pyrrolidinone-5-carboxylic acid, 5-Carboxy-2-pyrrolidinone, 5-Oxo-L-proline, 5-Oxoproline, 5-Pyrrolidinone-2-carboxylic acid, Glutimic acid, Glutiminic -acid, L-2-Pyrrolidone-5-carboxylic acid, L-5-Carboxy-2-pyrrolidinone, L-5-Oxo-2-pyrrolidinecarboxylic acid, L-5-Oxoproline, L-Glutamic acid, gamma-lactam, L-Glutimic acid, L-Glutiminic acid, L-Pyroglutamic acid, L-Pyrrolidinonecarboxylic acid, L-Pyrrolidonecarboxylic acid, Oxoproline, PCA, Pidolic acid, Pyroglutamic acid, Pyrrolidinonecarboxylic acid, Pyrrolidone-5-carboxylic acid, and Pyrrolidonecarboxylie acid.

The DL form of pyroglutamic acid (a mixture of the D and L stereoisomers) is known by the following names: DL-Proline, 5-oxo-(±)-2-Pyrrolidone-5-carboxylic acid, (±)-Pyroglutamic acid, 5-Oxo-DL-proline, DL-2-Pyrrolidinone-5-carboxylic acid, DL-2-Pyrrolidone-5-carboxylic acid, DL-Pyroglutamate, DL-Pyroglutamic acid, DL-Pyrrolidonecarboxylic acid, and Oxoproline. The DL form may be commercially available from Ajinomoto as AJIDEW A 100 and AJIDEW N 50 (Na-PCA).

Some of the above-listed stereoisomers are commercially available from UCIB, France via Barnet Products Corp., New Jersey. Such compounds are sold under trade names such as CUIVRIDONE (Cu-PCA) and L-FER PIDOLATE (Fe-PCA), and PIDOLIDONE.

Wherein the compositions herein comprise pyroglutamic acid, the compositions may comprise from about 0.01% to about 20%, alternatively from about 0.1% to about 10%, alternatively from about 0.25% to about 8%, and alternatively from about 0. 1% to about 5%, by weight of the composition.

### Optional Components of the Present Compositions

In addition to the guaiacol component, mucoadhesive polymer, and any acid necessary to achieve a pH of about 5.5 or less, the compositions herein may further comprise any of a variety of further optional components such as medicaments or carrier materials.

The optional components are desirable physically, and are chemically compatible with the essential components described hereinabove. Optional components suitable for use herein include medicaments and materials such as pH adjusting agents, chelating agents, preservatives, sweeteners, sensates, flavors, fragrances, volatile oils, mucilages, surfactant spreading aids including polyoxyethylene (20) sorbitan mono-oleate commercially sold as Polysorbate 80, and the like. Unless otherwise specified, the compositions may optionally comprise one or more given optional components at concentrations ranging from about 0.001% to about 20%, alternatively from about 0.01% to about 10%, by weight of the composition.

Non-limiting examples of certain optional components are as follows:

Metal Compound

The compositions of the present invention may optionally comprise a safe and effective amount of a metal compound. The metal compound is commonly referred to as a “metal salt”, wherein metal ion substituents such as iron, silver, copper, and zinc are believed to be primary components of a metal compound that provide for a hostile environment for common cold and influenza viruses.

Where present, the concentration of the metal compound in the compositions of the present invention may optionally range from about 0.001% to about 20%, preferably from about 0.01% to about 10%, more preferably from about 0.05% to about 5%, most preferably from about 0.05% to about 2%, by weight of the composition. The metal compound can be included in the compositions as an individual metal compound or as a combination of metal compounds, wherein the total concentration of metal compound may optionally range from about 0.001% to about 20% by weight of the composition.

As previously stated, one embodiment of the compositions of the present invention is wherein the compositions comprise a combination of a guaiacol component and mucoadhesive polymer at a pH of between about 3 to about 5.5, to which a metal compound may optionally be included. When the compositions comprise this combination of ingredients, including an organic acid, the organic acid is included at concentrations ranging from about 0.01% to about 5%, preferably from about 0.1% to about 2.5% by weight of the composition, wherein the metal compound is included at concentrations ranging from about 0.05% to about 10%, preferably from about 0.1% to about 2.5% by weight of the composition.

Alternatively or additionally, the compositions of the present invention may optionally comprise the metal compound and organic acid in combination with pyroglutamic acid. Without being limited by theory, it is believed that wherein the compositions of the present invention include the metal compound, organic acid, and pyroglutamic acid, the metal compound and pyroglutamic acid can form a metal-acid complex that can provide for a synergistic immediate and residual anti-viral effect. The metal compound can be combined with pyroglutamic acid to form a metal-acid complex prior to incorporation of the metal-acid complex into the compositions of the present invention. If the metal-acid complex is formed prior to inclusion in the compositions herein, the metal-acid complex is included at concentrations ranging from about 0.001% to about 20%, preferably from about 0.01% to about 10%, more preferably from about 0.1% to about 5%, by weight of the composition.

The metal compound suitable for use herein include those metal compounds containing a metal ion including but not limited to manganese (Mn), silver (Ag), zinc (Zn), tin (Sn), iron (Fe), copper (Cu), aluminum (Al), nickel (Ni), cobalt (Co), and mixtures thereof. Preferred metal compounds include those metal compounds which contain Cu, Fe, or Zn metal ions, or combinations thereof.

Nonlimiting examples of a metal compound suitable for use herein include the metal compounds referred to as salicylates, fumarates, benzoates, glutarates, lactates, citrates, malonates, acetates, glycolates, thiosalicylates, adipates, succinates, gluconates, aspartates, glycinates, tartarates, malates, maleates, ascorbates, chlorides, sulphates, nitrates, phosphates, fluorides, iodides, pidolates, and mixtures thereof. The acetates, ascorbates, chlorides, benzoates, citrates, gluconates, glutarates, lactates, malates, malonates, salicylates, succinates, sulphates, and mixtures thereof are preferred metal compounds.

Specific examples of a metal compound suitable for use herein include zinc acetate, zinc chloride, zinc ascorbate, zinc gluconate, zinc pidolate, zinc succinate, zinc sulphate, zinc chloride, and mixtures thereof. Zinc acetate is the most preferred metal compound.

Wherein the compositions of the present invention comprise a metal compound containing zinc ion, it is believed that the zinc ion provides for antiviral properties. Furthermore, it is known that metal ions such as iron, silver, copper, and zinc can provide antiviral properties for the prevention and treatment of common cold and influenza-like symptoms. Particularly, zinc and its possible effects on common colds has been extensively documented, The Handbook for Curing the Common cold, George A. Eby, published 1994, George Eby Research, Texas, USA. The mechanism of its action is thought to be multifactorial. Zinc ions have been shown to be both antiviral and antibacterial. They are believed to inhibit cleavage of rhinovirus polypeptides, preventing replication and formation of infective virions. Zinc ions may reduce the ability of rhinoviruses to penetrate cell membranes, partly by lowering expression of intercellular adhesion molecule ICAM. Zinc ions have also been shown to stimulate T-cell lyphocytes, including production of the natural antiviral, interferon-gamma. Zinc ions have also been shown to stabilize cell plasma membranes, protecting cells from cytotoxic agents, and preventing cell leakage.

Medicaments

Homeopathic and Non-homeopathic

The compositions of the present invention may also optionally comprise a homeopathic or non-homeopathic medicament. Homeopathic medicaments will be well-known to those of ordinary skill in the art. As examples, a detailed list of such homeopathic medicaments is found in The Homeopathic Pharmacopoeia of the United States, 2004 ed., published by The Homeopathic Pharmacopoeia of the U.S. Revision Service, as well as the German Homeopathic Pharmacopeia. Most countries have such a compendium of homeopathic medicaments, and the present invention is not limited to use of compounds listed in the Homeopathic Pharmacopoeia of the United States.

Likewise, the present invention is not limited only to homeopathic medicaments. Other, non-homeopathic preparations of the medicaments listed herein can also be used with the present invention, including, herbal, naturally-occurring, selectively bred, hybrid, synthetic or engineered materials.

As a non-limiting example, a homeopathic medicament usable with the present invention may be Echinacea. The genus Echinacea, a member of the sunflower family (Compositae or Asteracea) has nine species found in the U.S. and Canada (McGregor 1968). The three most common and widespread species, narrow-leafed purple coneflower (Echinacea angustifolia), pale purple coneflower (E. pallida) and purple cone flower (E. purpurea) have a long history of medicinal use, both in the United States and Europe. Echinacea is a botanical material that is believed to stimulate the immune system, and has traditionally been used to treat or prevent common colds, flu, and other respiratory infections.

Echinacea as used herein can be used in homeopathic or non-homeopathic preparations. Its use is not limited herein to any particular source or preparation and can include homeopathic preparations of E. purpurea or E. angustifolia, as well as extracts, isolations, purifications, concentrates and commercial preparations made from selectively bred plants, including but not limited to E. purpurea, E. angusifolia, E. pallida, or Pollinacea™ available from Indena, Milan, Italy. Extracts, isolations, purifications, concentrates and synthetic preparations of the active principals are also included. Active principals include but are not limited to echinacosides and/or polysaccharides.

Specific non-limiting examples of compositions of the present invention include compositions comprising Echinacea (including homeopathic or non-homeopathic preparations thereof) at any potency of 1× or lower, or concentrations ranging from about 1×10⁻²²% to about 1%, alternatively from about 1×10⁻²⁰% to about 0.5%, alternatively from about 1×10⁻¹⁹% to about 0.25%, and alternatively from about 1×10⁻⁷% to about 0.1% by weight of the composition.

Additional non-limiting examples of homeopathic medicaments that may optionally be used herein include: Echinacea (such as echinacea angustifolia or echinacea purpurea), magnesia muriatica (magnesium chloride), natrum muriaticum (sodium chloride), aconitum napellus, allium cepa, ammonium muriaticum, astragalus menziesii, atropinum, atropinum sulphuricum, baptisia tinctoria, berberinum, calcarea carbonica, calcarea muriatica, camphora, chininum muriaticum, chlorpromazinum, croton tiglium, dioscorea villosa, echinacea angustifolia, echinacea pallida, gelsemium sempervirens, helianthus annus, hepar sulphuris calcareum, histaminum hydrochloricum, hydrastininum muriaticum, hydrastis canadensis, hyoscyaminum, hyoscyaminum hydrobromatum, kali bromatum, kali carbonicum, kali iodatum, kali muriaticum, morphinum muriaticum, pulsatilla, pulsatilla nuttalliana, sambucus canadensis, sambucus nigra, scopolaminum hydrobromidum, spigelia marilandica, stachys betonica, sticta pulmonaria, thuja occidentalis, thymolum, thymus serpyllum, zincum aceticum, zincum bromatum, zincum carbonicum, zincum gluconicum zincum muriaticum, zincum oxydatum, zincum phosphoratum, zincum sulphuricum, and zingiber officinale, and mixtures thereof.

Allopathic Medicaments

The compositions of the present invention may also optionally comprise one or more allopathic medicaments. A detailed, but not necessarily a complete list, of allopathic or otherwise effective, optional components suitable for use with the compositions of the present invention are described in more detail hereinbelow.

Some non-limiting examples of types of allopathic medicaments usable with the present invention include decongestants, anticholinergics, anti-inflammatories, antivirals, and mucolytic compounds.

Example decongestants include: oxymetazoline, phenylephrine, xylometazoline, naphazoline, 1-desoxyephedrine, ephedrine, propylhexedrine, pseudoephedrine, and phenylpropanolamine.

Example anticholinergics include: ipratropium, chlorpheniramine, brompheniramine, diphenhydramine, doxylamine, clemastine, and triprolidine. Example anti-inflammatories include: ibuprofen, ketoprofen, diclofenac, naproxen, acetaminophen, and aspirin. Example antivirals include: amantidine, rimantidine, pleconaril, zanamivir, and oseltamivir. Example mucolytics include: acetylcysteine.

Sensates

In addition to the guaiacol component utilized herein, the compositions herein may optionally comprise a sensate. Sensates are commonly known in the art as components that provide a sensory effect. Sensory effects may include tingling, warming, and cooling sensations. Chemistry and Technology of Flavors and Fragrances, ed. D. J. Rowe, CRC Press, 2005.

Sensates that may be utilized will be commonly known. Non-limiting examples include menthol, camphor, eucalyptol, thymol, carvacrol, L-Carvone, OPTABREEZE®, and the like.

Wherein a sensate is utilized herein, the compositions may optionally comprise from about 0.001% to about 0.1% of the sensate, by weight of the composition.

Pharmaceutically Acceptable Carriers and Other Optional Components

The compositions of the present invention may be administered to respiratory tract or other mucosal areas as compositions comprising a pharmaceutically acceptable carrier system. Any pharmaceutically acceptable carrier in the form of a liquid, solid, or gas is suitable for the delivery of the compositions to prevent and treat common cold and influenza-like symptoms.

Depending on the desired dose form of the composition and, where applicable, the delivery device to be used, the compositions of the present invention may optionally include a pharmaceutically acceptable carrier such as water, water-miscible solvents including ethanol, propylene glycol, polyethylene glycol, transcutol, glycerol, and other known or otherwise effective water-miscible solvents; liquid aerosol propellants; and mixtures thereof. In one embodiment, these carriers are isotonic with human plasma.

When the compositions of the present invention are administered using water as a pharmaceutically acceptable carrier, the water may be purified or de-ionized water, and is substantially free of organic impurities and/or meets the USP guidelines for purified water. The concentration of water utilized to formulate the compositions into a final product form for delivery to respiratory tract areas ranges from about 40% to about 99.98%, preferably from about 80% to about 99.95%, by weight of the final product formulation.

When the compositions of the present invention are administered using a solid pharmaceutically acceptable carrier, the carrier may be applied in a powder form. In other words, the compositions of the present invention can be applied as a solid powder containing the essential ingredients and any optional components described herein with or without any known or otherwise effective solidification aids. However, pharmaceutically acceptable solid carriers can be added to provide aid in processing of the compositions, to aid in the consistency of the compositions, to provide for improved stability, to facilitate handling, for hygroscopicity benefits, and so forth. Pharmaceutically acceptable solid carrier materials include ingredients such as particulate and powder fillers, for example, a lactose powder, a sucrose powder and/or mixtures thereof. For respiratory tract compositions in the form of nasal compositions that are administered using a solid powder pharmaceutically acceptable carrier, the particle size of the powder is typically greater than 10 microns, especially when the nasal composition is a nasal inhalant.

As another non-limiting example, the composition may comprise a sweetener. Some natural and artificial sweeteners usable with the present invention include but are not limited to glucose, fructose, saccharine and its salts, sucrose, cyclamates, xylatols, ace sulfane K, sucralose, and aspartame.

In one embodiment, the compositions herein have a pH of about 5.5 or less, and alternatively about 4.5 or less. A specific nonlimiting example of another optional component suitable for use in the present invention include optional pH adjusting agents. Such optional pH adjusting agents include those normally associated with use in nasal compositions including compounds such as sodium bicarbonate, sodium phosphate, sodium hydroxide, ammonium hydroxide, sodium stannate, triethanolamine, sodium citrate, disodium succinate, and mixtures thereof. If present, the optional pH adjusting agents are generally included at concentrations ranging from about 0.01% to about 5.0% by weight of the composition.

Another specific nonlimiting example of an optional component suitable for use in the present invention includes chelating agents which are believed to provide for enhanced antiviral activity. Optional chelating agents useful in the compositions of the present invention include those that chelate transition metal ions such as iron, copper, zinc and other such metals. Not to be bound by theory, it is reasonable to postulate that metal ions, specifically metal cations, play a major role in the formation of oxidizing species. Oxidizing reactions and free radical formation can contribute to cellular damage in inflammatory diseases. The optional chelating agents useful herein are known to dampen oxidation reactions. The optional chelating agents are stable and effective in non-aqueous and aqueous mediums, and at pH ranges between about 3 to about 6.

Nonlimiting examples of suitable optional chelating agents include physic acid, sodium and calcium salts of ethylene diamine tetraacetic acid (EDTA), tetrasodium EDTA, sodium hexametaphosphate (SHMP), di(hydroxyethyl)glycine, 8-hydroxyquinoline, and mixtures thereof.

Wherein the compositions of the present invention comprise one or more optional chelating agents, the chelating agents are included at concentrations ranging from about 0.001% to 10%, preferably from about 0.005% to about 5%, more preferably from about 0.01% to about 2%, by weight of the composition.

Additionally, the present invention may optionally include one or more antioxidant compounds to reduce or combat free radical formation. Some nonlimiting examples of antioxidants usable in the present invention include vitamin E, BHT, BHA, benzoic acid, and ascorbic acid.

Other nonlimiting examples of optional components include certain salts of USP grade. Such salts provide enhanced compatibility with nasal cavity tissues. Nonlimiting examples include sodium chloride, magnesium chloride and mixtures thereof. However, many sodium, potassium and other salts can be used. Depending on the formulation, total salt concentration can range from about 0.0001% to about 1.0%, and alternatively from about 0.01% to about 0.5%, and alternatively from about 0. 1% to about 0.5%.

Other specific nonlimiting examples of optional components suitable for use in the present invention include preservatives. One or more preservatives can optionally be included to prevent microbial contamination that can be attributed to dosing devices of the composition applied to the nose. Such optional preservatives include those normally associated with use in nasal compositions including benzalkonium chloride, chlorhexidine gluconate, phenyl ethyl alcohol, I phenoxyethanol, benzyl alcohol, sorbic acid, thimerosal, phenylmercuric acetate, benzoates, parabens, sorbates, and mixtures thereof. Particularly, benzalkonium chloride is effective at pH's of about 3.5 to 5.5. Generally, the effectiveness of preservatives decreases as pH decreases. Therefore, benzalkonium chloride is particularly useful in the present invention because of its effectiveness at lower pH.

Method of Making

The compositions of the present invention may be prepared by any known or otherwise effective techniques suitable for providing a composition that provides a therapeutic benefit in the prevention and treatment of common cold and influenza-like symptoms. The compositions are preferably formulated to comprise a guaiacol component and mucoadhesive polymer, wherein the compositions have a pH of about 5.5 or less, optionally with a medicament described herein, wherein these compositions are then manufactured into final product forms of liquids, sprays, powders, inhalants, drops, or the like for administration to respiratory areas to prevent or treat symptoms associated with respiratory tract viral infections.

As a non-limiting example, the following procedure may be utilized. The compositions exemplified herein below in Table II may be prepared by solubilizing a mucoadhesive polymer in a liquid carrier such as water. While stirring, pyroglutamic acid, organic acid (for example, succinic acid) and salt-mix are then added to the polymer solution. Next, a premix, containing the guaiacol component and various optional components such as a sensate, is added to the cooled solution with continued stirring. The pH of the resultant solution may be between about 3 and about 5.5, however, a pH adjusting agent such as hydrochloric acid, sodium hydroxide and/or disodium succinate can be added to maintain the pH of the resultant solution to values of about 4.5 or less. These final compositions are suitable for incorporation into fill dropper vials, wherein the compositions are sprayed into a respiratory tract area such as the nostrils or turbinates for effective prevention and treatment of common cold and influenza-like symptoms. Typically, about 50-130 microliters of the composition are sprayed into each nostril or turbinate.

When the nonlimiting example compositions of the present invention are administered using a pharmaceutically acceptable carrier such as a powder, the compositions may be prepared by dry blending pyroglutamic acid and an organic acid using a mixer. A pH adjusting agent such as sodium citrate can be added to the dry blend. The dry blend is then micronized using a fluid energy mill. The resultant micronized dry blend is then dry mixed with a powder filler such as lactose powder, sucrose powder and/or mixtures thereof. This final powder composition can optionally be coated with a guaiacol component and/or a sensate mix using known spray coating techniques. The final powder composition can be filled into a nasal inhalation metering pump to prevent and treat symptoms of the common cold and influenza, wherein about 10 milligrams (mgs) of the final powder can be administered to a respiratory tract area such as a nostril or a turbinate.

As stated herein, the compositions of the present invention are suitable for administration in final nonlimiting product forms of liquids, sprays, inhalants, powders, and so forth. Suitable devices utilized in the administration of these final compositions include those commonly employed or otherwise effective liquid containers, droppers, spray containers including pressurized sprayers, pumps and pump containers, inhalation devices, powder containers, atomizers, and so forth.

Homeopathic Ingredient Method of Making

The Homeopathic Pharmacopoeia (HPUS-Dec 2004 ed. of the HPUS Revision Service) is the reference book of standards for source, composition, identity and specifications for preparation of homeopathic medicines (drugs). Homeopathic drugs comprise active ingredients from the HPUS formulated into “tinctures” and “attenuations”. In the present invention, as a non-limiting example, three such active ingredient tinctures and attenuations are prepared for inclusion in the manufacture of the present invention, under the guidelines for homeopathic medicines: Echinacea attenuation (6×), Sodium chloride attenuation (3×) and Magnesium chloride attenuation (3×). Once prepared, the attenuations are incorporated into the present invention as a typical raw material ingredient. Starting or “Mother” tinctures maybe obtained commercially from a variety of sources familiar with the HPUS or prepared by the formulator.

(Mother) Tincture Preparation

A 1/10 (10%) solution corresponds to a 1× liquid attenuation. A 1× or 10% solution is known in the homeopathic art as a “Mother Tincture”. Subsequent dilutions are known as “attenuations”. One milliliter (1.0 ml) of 1× liquid attenuation is “succussed” or violently shaken, with nine milliliters (9.0 ml) of diluent to produce ten (10.0 ml) of a 2× attenuation. The diluent may be an alcohol/water mixture or aqueous solution depending on the particular monographed requirments in the HPUS. Subsequent attenuations are prepared by succussing 1.0 ml of the preceding attenuation in 9.0 ml of diluent to produce 10.0 ml of the succeeding attenuation.

Echinacea Attenuation Preparation

From 10% “Mother” Tincture (a 1× or 10% solution): One milliliter (1.0) of 10% mother tincture of Echinacea purpurea or Echinacea angustifolia (hereinafter referred to as “Echinacea”) is measured out by volume and diluted with nine (9.0) milliliters of USP water and succussed ten times to produce ten milliliters of 2× attenuation. One (1.0) milliliter of 2× attenuation is diluted with nine milliliters of USP water, and succussed ten times to produce 10 milliliters of 3× attenuation. This procedure is repeated until a 6× attenuation is achieved via a total of 6 dilutions (0.0001%). The Echinacea 6× attenuation is added to the main batch in a quantity necessary to achieve the desired final concentration of Echinacea.

From Dry Extract: Weigh-out 1.0 gram of Echinacea dry extract and add nine (9.0) milliliters of 55% ethyl alcohol. Succuss ten times to arrive at the Mother Tincture. Measure by volume, one milliliter of 10% (1×) tincture, and add nine milliliters of USP water. Succuss ten times to produce 10 milliliters of 2× attenuation. Measure one (1.0) milliliter of 2× attenuation, and add nine milliliters of USP water, succuss ten times to produce ten milliliters of 3× attenuation. Repeat this procedure to produce a 6× attenuation. A portion of Echinacea 6× tincture is added to the final batch in a quantity necessary to achieve the desired final concentration of Echinacea.

Sodium Chloride 3× Attenuation

Weigh-out 15 grams of USP sodium chloride, and add 135 grams of USP water, succuss ten times in a closed vessel to produce ˜150 milliliters of IX “Mother” Tincture. Add 1,250 milliliters of USP water to 150 grams of the 1× tincture. Succuss ten times to produce ˜1,500 milliliters of 2× attenuation. Add 13,500 milliliters of USP water to 1,500 milliliters of 2× attenuation, succuss ten times to produce 15,000 milliliters of 3× attenuation. Add a sufficient quantity of sodium chloride 3× attenuation to the final batch necessary to achieve the desired final concentration of sodium chloride.

Magnesium Chloride Hexahydrate 3× Attenuation

Weigh-out 15 grams of USP magnesium chloride hexahydrate and add 135 grams of 70% ethyl alcohol, succuss ten times in a closed vessel to produce ˜150 milliliters of 1× Mother Tincture. Add 1,250 milliliters of USP water to 150 milliliters of 1× tincture, succuss ten times to produce ˜1,500 milliliters of 2× attenuation. Add 13,500 milliliters of USP water to 1,500 milliliters of 2× attenuation to produce 15,000 milliliters of 3× attenuation. Add a sufficient quantity of magnesium chloride hexahydrate 3× attenuation to the final batch to achieve the desired final concentration of magnesium chloride.

Non Homeopathic Method of Making

Alternatively, nonlimiting example compositions of the present invention may be made by conventional pharmaceutical processing procedures. For example, the polysorbate 80 surfactant is dissolved in USP water, with agitation and slight heating if necessary, followed by cooling to facilitate the addition of mucoadhesive polymer (for example, Lutrol F127), sweetener(s) and medicament(s) (for example, Echinacea extracts such as Pollinacea™ available from Indena, Milan, Italy). In a separate vessel, PCA, organic acid(s) and salt(s) are added sequentially to heated water (<50C) with agitation and then added to the polymer solution. In a third separate vessel flavor(s), sensate(s) and guaiacol component(s) are premixed, and added to the main mix which has been cooled to <20C. The resulting combined mix is covered and agitated for 15-20 min at 200-400 rpm, or until well mixed. The resulting example product has a pH of 3.3 to 3.7, and a viscosity <100 cps.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified concentrations are weight-weight percents, unless otherwise specified.

Exemplary compositions of the present invention are exemplified in Table I herein below. These compositions may also comprise a premix containing optional ingredients such as flavors, fragrances, sensates, and the like. The premix also comprises a guaiacol component, as exemplified in Table II herein below. The exemplified premixes of Table II provide for compositions that are aesthetically pleasing in taste, flavor, coolness, smell, and the like and that, due to the guaiacol component, reduce the irritation of the nasal cavity upon application and improve the stability of the composition at the low pH of the composition.

The nonlimitng example compositions exemplified herein below in Table II may be prepared in accordance with the guidance provided hereinabove.

TABLE I

| | Premix Mix A | Premix Mix B | Premix C | Premix D | Premix E | Premix F | Premix G |
|---|---|---|---|---|---|---|---|
| Component | (Wt %) | (Wt %) | (Wt %) | (Wt %) | (Wt %) | (Wt %) | (Wt %) |
| Ethanol | 54.5 | | | | | | |
| Menthol | 6.0 | 13.3 | 30.5 | 28.8 | 53.3 | 12.5 | 3.2 |
| Eucalyptol | 2.5 | 5.5 | 39.0 | 9.8 | 5.7 | 4.0 | 1.4 |
| Camphor | | | | | 10.0 | | |
| L-Carvone | | | 30.5 | 7.7 | | | 1.5 |
| Optabreeze A | 14.7 | 32.2 | | | | 22.7 | |
| Eugenol | 22.3 | 49.0 | | 53.7 | 31.0 | 34.5 | 6.5 |
| Propenyl Guaethol | | | | | | 26.3 | |
| Clove Oil | | | | | | | 87.4 |
| Total: | 100 | 100 | 100 | 100 | 100 | 100 | 100 |

TABLE II

| | Sample I | Sample 2 | Sample 3 | Sample 4 |
|---|---|---|---|---|
| Component | (Wt %) | (Wt %) | (Wt %) | (Wt %) |
| Pyroglutamic | 0.36 | 0.36 | 0.36 | 0.36 |
| Acid | | | | |
| Succinic Acid | 1.04 | 1.04 | 1.04 | 1.04 |
| Disodium | 0.47 | 0.47 | 0.47 | 0.47 |
| Succinate | | | | |
| Sodium | 0.21 | 0.21 | 0.21 | 0.21 |
| Chloride | | | | |
| Magnesium | | | | |
| Chloride | | | | |
| Polysorbate 80 | 0.08 | 0.08 | 0.08 | 0.08 |
| Lutrol F-127 | 16 | | | |
| Sodium | 0.03 | 0.03 | 0.03 | 0.03 |
| Saccharin | | | | |
| Hydroxypropyl | | 1.00 | 1.00 | 1.00 |
| methyl | | | | |
| cellulose E4M | | | | |
| Benzalkonium | 0.3 | 0.3 | 0.3 | 0.3 |
| Chloride (50%) | | | | |
| Echinacea | 0.3 | 0.3 | 0.01 | 0.01 |
| Premix | A | D | D | D |
| Atropine | | | 0.0001 | |
| Sulphate | | | | |
| Scopalmine | | | | 0.0001 |
| HBr | | | | |
| USP Water | qs 100% | qs 100% | qs 100% | qs 100% |

| | Sample 4 | Sample 5 | Sample 6 | Sample 7 |
|---|---|---|---|---|
| Component | (Wt %) | (Wt %) | (Wt %) | (Wt %) |
| Pyroglutamic | 0.36 | 0.36 | 0.36 | 0.36 |
| Acid | | | | |
| Succinic Acid | 1.04 | 1.04 | 1.04 | 1.04 |
| Disodium | 0.47 | 0.47 | 0.47 | 0.47 |
| Succinate | | | | |
| Sodium | 0.21 | 0.21 | 0.21 | 0.10 |
| Chloride | | | | |
| Magnesium | | | | 0.10 |
| Chloride | | | | |
| hexahydrate | | | | |
| Polysorbate 80 | 0.08 | 0.08 | 0.08 | 0.08 |
| Lutrol F-127 | 16 | | 15 | 15 |
| Sodium | 0.03 | 0.03 | 0.03 | 0.03 |
| Saccharin | | | | |
| Hydroxypropyl | | 1.00 | 1.00 | 1.00 |
| methyl | | | | |
| cellulose E4M | | | | |
| Benzalkonium | 0.3 | 0.3 | 0.3 | 0.3 |
| Chloride(50%) | | | | |
| Echinacea | 0.01 | 0.01 | 0.01 | 1.5 × 10⁻¹⁷ |
| Premix | D | D | B | A |
| Atropine | 0.0001 | 0.0001 | | |
| Sulphate | | | | |
| Scopalmine | 0.0001 | | | |
| HBr | | | | |
| EDTA | | 0.26 | | |
| USP Water | qs 100% | qs 100% | qs 100% | qs 100% |

| | Sample 8 | Sample 9 | Sample 10 |
|---|---|---|---|
| Component | (Wt %) | (Wt %) | (Wt %) |
| Pyroglutamic | 0.36 | 0.36 | 0.36 |
| Acid | | | |
| Succinic Acid | 1.04 | 1.04 | 1.04 |
| Disodium | 0.47 | 0.47 | 0.47 |
| Succinate | | | |
| Sodium | 0.10 | 0.05 | 0.06 |
| Chloride | | | |
| Magnesium | 0.10 | 0.05 | 0.10 |
| Chloride | | | |
| hexahydrate | | | |
| Polysorbate 80 | 0.08 | 0.08 | 0.08 |
| Lutrol F-127 | 15 | 15 | 15 |
| Sodium | 0.03 | 0.03 | 0.03 |
| Saccharin | | | |
| Hydroxypropyl | | | |
| methyl | | | |
| cellulose E4M | | | |
| Benzalkonium | 0.3 | 0.3 | 0.3 |
| Chloride(50%) | | | |
| Echinacea | | 5 × 10⁻⁵ | 1 × 10⁻³ |
| Premix | A | A | F |
| Atropine | | | |
| Sulphate | | | |
| Scopalmine | | | |
| HBr | | | |
| Vitamin E | 1.00 | | |
| EDTA | | | |
| USP Water | qs 100% | qs 100% | qs 100% |

Wt. %—Weight Percent
1-pyroglutamic acid available from UCIB, France via Barnet Products Corp., New Jersey
2-succinic acid available from DSM Fine Chemicals, UK
3-Carbopol 980 available from B. F. Goodrich Company, USA
4-hydroxypropyl methylcellulose available from Colorcon Ltd., Kent, UK
5-Lutrol F-127 available from BASF Specialty Chemicals, Mount Olive, N.J., USA
6-Optabreeze A available from Symrise Corporation, Teterboro, N.J. USA

What is claimed is:

1. A composition comprising:
(a) a guaiacol component; and
(b) a mucoadhesive polymer;
wherein said composition has a pH of about 5.5 or less.

2. The composition of claim 1 further comprising a medicament.

3. The composition of claim 1 wherein said guaiacol component is selected from the group consisting of: eugenol, iso-eugenol, dihydroeugenol, vanillyl butyl ether, vanillin, 5-propenylguaethol, 4-ethyl-2-methoxyphenol, 4-allyl-2-methoxyphenol acetate, 4-methyl guaiacol and mixtures thereof.

4. The composition of claim 1 wherein said guaiacol component is in the range of from about 0.0001% to about 1% by weight of said composition.

5. The composition of claim 1 wherein said composition comprises an acid selected from the group consisting of: ascorbic acid, salicylic acid, fumaric acid, benzoic acid, glutaric acid, lactic acid, citric acid, malonic acid, acetic acid, glycolic acid, malic acid, adipic acid, succinic acid, aspartic acid, phthalic acid, tartaric acid, glutamic acid, gluconic acid, and mixtures thereof.

6. The composition of claim 1 wherein said mucoadhesive polymer is selected from the group consisting of: carboxypolymethylenes, carboxyvinyl polymers, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, natural polymers, polymeric thermoreversible polymers, ionic responsive polymers, copolymers of polymethyl vinyl ether and maleic anhydride, and mixtures thereof.

7. The composition of claim 6 wherein said mucoadhesive polymer is a cellulose derivative selected from the group consisting of hydroxypropyl methylcelluloses, hydroxypropyl celluloses, methyl cellulose polymers, carboxymethyl cellulose polymers, salts of carboxymethyl cellulose, and mixtures thereof.

8. The composition of claim 6 wherein said mucoadhesive polymer is a thermoreversible polymer selected from the group consisting of: poloxamers, ethylhydroxy ethylcelluloses, and mixtures thereof.

9. The composition of claim 1 wherein said composition has a viscosity of from about 1 cps to about 2000 cps.

10. The composition of claim 9 wherein said composition has a viscosity of from about 10 cps to about 600 cps.

11. The composition of claim 10 wherein said composition has a viscosity of from about 20 to about 500 cps.

12. The composition of claim 2 wherein wherein said medicament is selected from the group consisting of: echinacea purpurea, magnesia muriatica, natrum muriaticum, aconitum napellus, allium cepa, ammonium muriaticum, astragalus menziesii, atropinum, atropinum sulphuricum, baptisia tinctoria, berberinum, calcarea carbonica, calcarea muriatica, camphora, chininum muriaticum, chlorpromazinum, croton tiglium, dioscorea villosa, echinacea angustifolia, echinacea pallida, gelsemium sempervirens, helianthus annus, hepar sulphuris calcareum, histaminum hydrochloricum, hydrastininum muriaticum, hydrastis canadensis, hyoscyaminum, hyoscyaminum hydrobromatum, kali bromatum, kali carbonicum, kali iodatum, kali muriaticum, morphinum muriaticum, pulsatilla, pulsatilla nuttalliana, sambucus canadensis, sambucus nigra, scopolaminum hydrobromidum, spigelia marilandica, stachys betonica, sticta pulmonaria, thuja occidentalis, thymolum, thymus serpyllum, zincum aceticum, zincum bromatum, zincum carbonicum, zincum gluconicum zincum muriaticum, zincum oxydatum, zincum phosphoratum, zincum sulphuricum, and zingiber officinale, and mixtures thereof.

13. The composition of claim 2 wherein said medicament is Echinacea or an active principal thereof.

14. The composition of claim 2 wherein said medicament is selected from the group consisting of: oxymetazoline, phenylephrine, xylometazoline, naphazoline, 1-desoxyephedrine, ephedrine, propylhexedrine, pseudoephedrine, phenylpropanolamine, ipratropium, chlorpheniramine, brompheniramine, diphenhydramine, doxylamine, clemastine, triprolidine, ibuprofen, ketoprofen, diclofenac, naproxen, acetaminophen, aspirin, their salts, and mixtures thereof.

15. The composition of claim 1 further comprising a pH adjusting agent selected from the group consisting of: sodium bicarbonate, sodium phosphate, sodium hydroxide, ammonium hydroxide, sodium stannate, triethanolamine, sodium citrate, disodium succinate, and mixtures thereof.

16. The composition of claim 1 further comprising pyroglutamic acid.

17. The composition of claim 5 further comprising pyroglutamic acid.

18. The composition of claim 1 further comprising a metal compound.

19. The composition of claim 18 wherein said metal compound is a metal compound containing a metal ion selected from the group consisting of: manganese (Mn), silver (Ag), zinc (Zn), tin (Sn), iron (Fe), copper (Cu), aluminum (Al), nickel (Ni), cobalt (Co), and mixtures thereof.

20. The composition of claim 19 wherein said metal compound is selected from the group consisting of: salicylates, fumarates, benzoates, glutarates, lactates, citrates, malonates, acetates, glycolates, thiosalicylates, adipates, succinates, gluconates, aspartates, glycinates, tartarates, malates, maleates, ascorbates, chlorides, sulphates, nitrates, phosphates, fluorides, iodides, pidolates, and mixtures thereof.

21. The composition of claim 20 wherein said metal compound is selected from the group consisting of: zinc acetate, zinc chloride, zinc ascorbate, zinc gluconate, zinc pidolate, zinc succinate, zinc sulphate, zinc chloride, and mixtures thereof.

22. The composition of claim 21 wherein said metal compound is zinc acetate.

23. The composition of claim 1 wherein said composition is a nasal composition selected from the group consisting of: nasal liquids, nasal sprays, nasal inhalants, nasal powders, nasal drops, and mixtures thereof.

24. The composition of claim 23 wherein said nasal composition is a nasal spray.

25. A method of treating and preventing a symptom associated with an upper respiratory infection in a mammal in need of such treatment and prevention, comprising administering to the mammal a composition comprising a guaiacol component and a mucoadhesive polymer, wherein said composition has a pH of about 5.5 or less.

26. The method of claim 25 wherein said composition further comprises a medicament.

27. The method of claim 25 wherein said guaiacol component is selected from the group consisting of: eugenol, iso-eugenol, dihydroeugenol, vanillyl butyl ether, vanillin, 5-propenylguaethol, 4-ethyl-2-methoxyphenol, 4-allyl-2-methoxyphenol acetate, 4-methyl guaiacol, and mixtures thereof.

28. The method of claim 25 wherein said composition comprises at least one acid selected from the group consisting of ascorbic acid, salicylic acid, fumaric acid, benzoic acid, glutaric acid, lactic acid, citric acid, malonic acid, acetic acid, glycolic acid, malic acid, adipic acid, succinic acid, aspartic acid, phthalic acid, tartaric acid, glutamic acid, gluconic acid, and mixtures thereof.

29. The method of claim 25 wherein said mucoadhesive polymer is selected from the group consisting of carboxypolymethylenes, carboxyvinyl polymers, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, natural polymers, polymeric cellulose derivatives, polyvinyl pyrrolidones, dextran polymers, polyethylene oxide polymers, thermoreversible polymers, ionic responsive polymers, copolymers of polymethyl vinyl ether and maleic anhydride, and mixtures thereof.

30. The method of claim 25 wherein wherein said medicament is selected from the group consisting of: echinacea purpurea, magnesia muriatica, natrum muriaticum, aconitum napellus, allium cepa, ammonium muriaticum, astragalus menziesii, atropinum, atropinum sulphuricum, baptisia tinctoria, berberinum, calcarea carbonica, calcarea muriatica, camphora, chininum muriaticum, chlorpromazinum, croton tiglium, dioscorea villosa, echinacea angustifolia, echinacea pallida, gelsemium sempervirens, helianthus annus, hepar sulphuris calcareum, histaminum hydrochloricum, hydrastininum muriaticum, hydrastis canadensis, hyoscyaminum, hyoscyaminum hydrobromatum , kali bromatum, kali carbonicum, kali iodatum, kali muriaticum, morphinum muriaticum, pulsatilla, pulsatilla nuttalliana, sambucus canadensis, sambucus nigra, scopolaminum hydrobromidum, spigelia marilandica, stachys betonica, sticta pulmonaria, thuja occidentalis, thymolum, thymus serpyllum, zincum aceticum, zincum bromatum, zincum carbonicum, zincum gluconicum zincum muriaticum, zincum oxydatum, zincum phosphoratum, zincum sulphuricum, and zingiber officinale, and mixtures thereof.

31. The method of claim 25 wherein said medicament is Echinacea or an active principal thereof.

32. The method of claim 25 wherein said medicament is selected from the group consisting of: oxymetazoline, phenylephrine, xylometazoline, naphazoline, 1-desoxyephedrine, ephedrine, propylhexedrine, pseudoephedrine, phenylpropanolamine, ipratropium, chlorpheniramine, brompheniramine, diphenhydramine, doxylamine, clemastine, triprolidine, ibuprofen, ketoprofen, diclofenac, naproxen, acetaminophen, aspirin, their salts, and mixtures thereof.

33. The method of claim 25 wherein said composition further comprises pyroglutamic acid.

34. The method of claim 28 wherein said composition further comprises pyroglutamic acid.

35. The method of claim 25 wherein said composition has a viscosity of from about 1 cps to about 2000 cps.

36. The method of claim 25 wherein said composition is a nasal spray.

37. The method of claim 36 wherein said nasal spray comprises from about 40% to about 99.98% by weight of a pharmaceutically acceptable carrier selected from the group consisting of water, ethanol, propylene glycol, polyethylene glycol, transcutol, glycerol, a liquid aerosol propellant, and mixtures thereof.
